(19) United States
(12) Patent Application Publication
(10) Pub. No.: US 2025/0111934 A1
(43) Pub. Date: Apr. 3, 2025

(54) MEDICAL IMAGING SYSTEM AND CONTROL METHOD FOR MEDICAL IMAGING SYSTEM

(71) Applicant: GE Precision Healthcare LLC, Waukesha, WI (US)

(72) Inventors: Minahui Ye, Beijing (CN); Bingkun Xue, Beijing (CN); Yingying Wang, Beijing (CN); Yuqing Li, Beijing (CN); Yichen Wang, Beijing (CN); Yannan Huang, Beijing (CN)

(21) Appl. No.: 18/900,213

(22) Filed: Sep. 27, 2024

(30) Foreign Application Priority Data
Sep. 28, 2023 (CN) 202311282013.8

(51) Int. Cl. G16H 30/40; G06T 1/00

(52) U.S. Cl. CPC G16H 30/40; G06T 1/0007

(57) ABSTRACT

Embodiments of the present application provide a medical imaging system and a control method for a medical imaging system. The system includes: an imaging apparatus, which acquires a medical image of a subject under examination; a non-contact detection apparatus, which performs non-contact detection on the subject under examination on the basis of a wireless signal, so as to acquire data related to vital signs of the subject under examination; a signal processing module, which processes the data to generate information reflecting the vital signs; and a display apparatus, which displays a graphical user interface used to control the imaging apparatus, and the information reflecting the vital signs. The present application helps to accurately determine an imaging timing.

### CROSS-REFERENCE TO RELATED APPLICATIONS

This application is based on and claims priority to Chinese Application No. 202311282013.8, filed on Sep. 28, 2023, the entire contents of which is herein incorporated by reference.

### TECHNICAL FIELD

Embodiments of the present application relate to the technical field of medical imaging, and relate in particular to a medical imaging system and a control method for a medical imaging system.

### BACKGROUND

In a medical imaging system, emitted X-rays from an X-ray source are directed at a subject under examination and are received by a detector after penetrating the subject under examination. The detector is divided into an array of discrete elements (such as pixels). The detector elements are read to produce an output signal on the basis of the amount or intensity of radiation impinging on each pixel region. The signal is processed to produce a medical image of the subject under examination, and the medical image can be displayed in a display apparatus of the medical imaging system.

### SUMMARY

When a medical imaging system is used to perform medical imaging on a subject under examination, imaging timing is a key factor for acquiring a high-quality medical image.

For example, when X-ray examination is performed on the chest or abdomen, exposure needs to be performed at an appropriate timing when the subject under examination has the maximal inspiratory volume, and if the exposure is performed at another timing, the acquired medical image sometimes cannot clearly show a site to be examined.

In order to accurately provide the imaging timing, embodiments of the present application provide a medical imaging system and a control method for a medical imaging system.

According to an aspect of an embodiment of the present application, there is provided a medical imaging system. The system includes:

An imaging apparatus, which acquires a medical image of a subject under examination;

A non-contact detection apparatus, which performs non-contact detection on the subject under examination on the basis of a wireless signal, so as to acquire data related to vital signs of the subject under examination;

A signal processing module, which processes the data to generate information reflecting the vital signs; and

A display apparatus, which displays a graphical user interface used to control the imaging apparatus, and the information reflecting the vital signs.

According to an aspect of an embodiment of the present application, a control method for a medical imaging system is provided. The medical imaging system has an imaging apparatus used to acquire a medical image of a subject under examination. The method includes:
Performing non-contact detection on the subject under examination on the basis of a wireless signal, so as to acquire data related to vital signs of the subject under examination;
Processing the data to generate information reflecting the vital signs; and
Displaying a graphical user interface used to control the imaging apparatus, and the information reflecting the vital signs.

With reference to the following description and drawings, specific implementations of the embodiments of the present application are disclosed in detail, and the means by which the principles of the embodiments of the present application can be employed are illustrated. It should be understood that the embodiments of the present application are therefore not limited in scope. Within the scope of the spirit and clauses of the appended claims, the embodiments of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive effort. In the drawings:

FIG. 1 is a schematic diagram of an X-ray imaging system according to an embodiment of the present application;

FIG. 2 is a schematic diagram of a medical imaging system according to an embodiment of the present application;

FIG. 3 is a schematic diagram in which a non-contact detection apparatus performs detection on a subject under examination;

FIG. 4 is a schematic diagram of information reflecting vital signs that is displayed by a display apparatus;

FIG. 5 is another schematic diagram of information reflecting vital signs that is displayed by a display apparatus;

FIG. 6 is yet another schematic diagram of information reflecting vital signs that is displayed by a display apparatus;

FIG. 7 is a schematic diagram of information reflecting vital signs that is displayed on a graphical user interface;

FIG. 8 is a schematic diagram of information on a time point at which an imaging apparatus acquires a medical image that is displayed by a display apparatus;

FIG. 9 is a schematic diagram of a processing module;

FIG. 10 is a schematic diagram in which information reflecting vital signs within a second time period is predicted on the basis of a long short-term memory neural network;

FIG. 11 is a schematic diagram of extreme values; and

FIG. 12 is a schematic diagram of a control method for a medical imaging system according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” and so on are used to distinguish different elements from one another by title, but do not represent the spatial arrangement, temporal order, or the like of the elements, and the elements should not be limited by said terms. The term “and/or” includes any one of and all combinations of one or more associated listed terms. The terms “comprise”, “include”, “have”, etc., refer to the presence of stated features, elements, components, or assemblies, but do not exclude the presence or addition of one or more other features, elements, components, or assemblies. The terms “connect”, “connected”, “couple”, as well as other similar terms to which the embodiments of the present application relate are not limited to physical or mechanical connections, but may include electrical connections, whether directly connected or indirectly connected.

In the embodiments of the present application, the singular forms “a” and “the” or the like include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ”, and the term “based on” should be construed as “at least in part based on . . . ”, unless otherwise clearly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

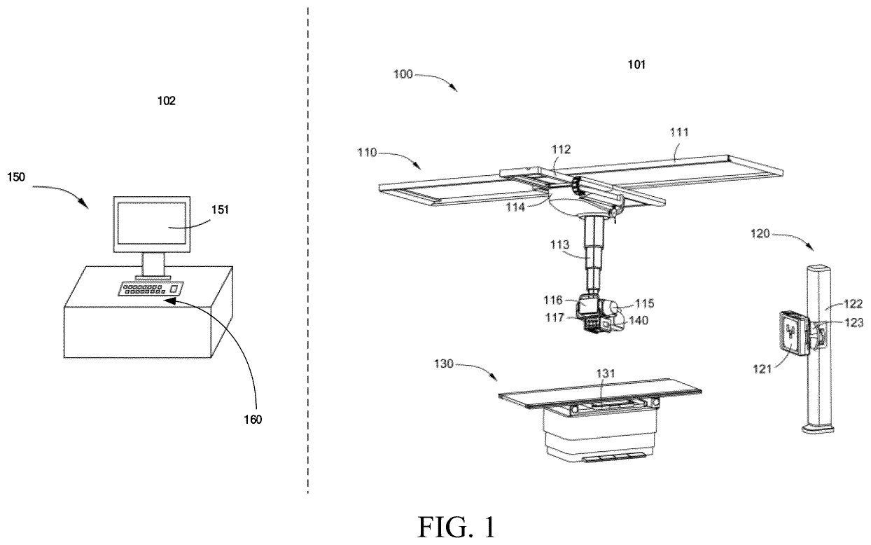

FIG. 1 is a schematic diagram of an X-ray imaging system according to an embodiment of the present application. As shown in FIG. 1, the X-ray imaging system 100 may be an example of a medical imaging system, which includes a suspension apparatus 110, a wall stand apparatus 120 and an examination table apparatus 130 provided in a scanning room 101, and a control apparatus 150 provided in a control room 102. The suspension apparatus 110 includes a longitudinal guide rail 111, a transverse guide rail 112, a telescopic cylinder 113, a sliding member 114 and a tube assembly 115.

Although some embodiments of the present application are described on the basis of a suspended X-ray imaging system, the embodiments of the present application are not limited thereto. For example, the medical imaging system may also be another type of X-ray imaging system; alternatively, the medical imaging system may also be another type of imaging system, such as a computerized tomography (CT) system, a positron emission tomography (PET) system, a magnetic resonance imaging (MRI) system, etc.

For ease of description, in the present application, an x-axis, y-axis, and z-axis are defined as the x-axis and the y-axis being located in a horizontal plane and being perpendicular to one another, and the z-axis being perpendicular to the horizontal plane. Specifically, the direction in which the longitudinal guide rail 111 is located is defined as the x-axis, the direction in which the transverse guide rail 112 is located is defined as the y-axis direction, and the direction of extension of the telescopic cylinder 113 is defined as the z-axis direction, and the z-axis direction is the vertical direction.

The longitudinal guide rail 111 and the transverse guide rail 112 are perpendicularly arranged, wherein the longitudinal guide rail 111 is mounted on a ceiling, and the transverse guide rail 112 is mounted on the longitudinal guide rail 111. The telescopic cylinder 113 is used to carry the tube assembly 115.

The sliding member 114 is disposed between the transverse guide rail 112 and the telescopic cylinder 113. The sliding member 114 may include components such as a rotary shaft, a motor, and a reel. The motor can drive the reel to rotate around the rotary shaft, which in turn drives the telescopic cylinder 113 to move along the z axis and/or slide relative to the transverse guide rail. The sliding member 114 can slide relative to the transverse guide rail 112, that is, the sliding member 114 can drive the telescopic cylinder 113 and/or the tube assembly 115 to move in the y-axis direction. Furthermore, the transverse guide rail 112 can slide relative to the longitudinal guide rail 111, which in turn drives the telescopic cylinder 113 and/or the tube assembly 115 to move in the x-axis direction.

The telescopic cylinder 113 includes a plurality of columns having different inner diameters, and the plurality of columns may be sleeved sequentially from bottom to top in columns located thereon to thereby achieve telescoping. The telescopic cylinder 113 can be telescopic (or movable) in the vertical direction, that is, the telescopic cylinder 113 can drive the tube assembly to move along the z-axis direction. The lower end of the telescopic cylinder 113 is further provided with a rotating part, and the rotating part may drive the tube assembly 115 to rotate.

The tube assembly 115 includes an X-ray tube, and the X-ray tube may produce X-rays and project the X-rays to a patient's intended region of interest (ROI). Specifically, the X-ray tube may be positioned adjacent to a beam limiter, and the beam limiter is used to align the X-rays with the patient's intended region of interest. At least part of the X-rays may be attenuated by means of the patient and may be incident on a detector 121/131.

The suspension apparatus 110 further includes a beam limiter 117. The beam limiter 117 is usually mounted below the X-ray tube. The X-rays emitted by the X-ray tube irradiate on the body of a subject under examination by means of an opening of the beam limiter 117. The size of the opening of the beam limiter 117 determines an irradiation range of the X-rays, namely, the size of a region of an exposure field of view (FOV). The positions of the X-ray tube and beam limiter 117 in the transverse direction determine the position of the exposure FOV on the body of the subject under examination. It is well known that X-rays are harmful to the human body, so it is necessary to control the X-rays so that the same only irradiate a site of the subject under examination that needs to be examined, namely, a region of interest (ROI).

The suspension apparatus 110 further includes a tube control apparatus (console) 116. The tube control apparatus 116 is mounted on the tube assembly. The tube control apparatus 116 includes user interfaces such as a display screen and a control button for performing preparation work before image capture, such as patient selection, protocol selection, positioning, etc.

The movement of the suspension apparatus 110 includes the movement of the tube assembly along the x-axis, y-axis, and z-axis, as well as the rotation of the tube assembly in a horizontal plane (the axis of rotation is parallel to or coincides with the z-axis) and in a vertical plane (the axis of rotation is parallel to the y-axis). In the described movement, a motor is usually used to drive a rotary shaft which in turn drives a corresponding component to rotate, so as to achieve a corresponding movement or rotation, and a corresponding control component is generally mounted in the sliding member 114. An X-ray imaging unit further includes a motion control unit (not shown in the figure), and the motion control unit can control the described movement of the suspension apparatus 110. Furthermore, the motion control unit can receive a control signal to control a corresponding component to move correspondingly.

The wall stand apparatus 120 includes a first detector assembly 121, a wall stand 122, and a connecting portion 123. The connecting portion 123 includes a support arm that is vertically connected in the height direction of the wall stand 122 and a rotating bracket that is mounted on the support arm, and the first detector assembly 121 is mounted on the rotating bracket. The wall stand apparatus 120 further includes a detector driving apparatus that is provided between the rotating bracket and the first detector assembly 121. Under the drive of the detector driving apparatus, the first detector assembly 121 moves along a direction that is parallel to the height direction of the wall stand 122 in a plane that is supported by the rotating bracket, and the first detector assembly 121 may be further rotated relative to the support arm to form an angle with the wall stand. The first detector assembly 121 has a plate-like structure the orientation of which can be changed, so that the incident surface of the X-rays becomes vertical or horizontal depending on the incident direction of the X-rays.

A second detector assembly 131 is included on the examination bed apparatus 130, and the selection or use of the first detector assembly 121 and the second detector assembly 131 may be determined on the basis of an image capture site of a patient and/or an image capture protocol, or may be determined on the basis of the position of the subject under examination that is obtained by the capturing of a camera, so as to perform image capture and examination at a supine, prone, or standing position. FIG. 1 merely shows a schematic diagram of a wall stand and an examination bed. It should be understood by those skilled in the art that a wall stand and/or examination bed in any form or arrangement may be selected or just the wall stand can be mounted, and the wall stand and/or examination bed do/does not limit the entire solution of the present application.

In some embodiments, the medical imaging system includes an image capture apparatus 140 (such as a camera). The subject under examination may be captured by the image capture apparatus to obtain a captured image that includes the subject under examination, for example a static optical image or a series of optical image frames in a dynamic real-time video stream, to carry out auxiliary positioning, exposure configurations, and the like. The image capture apparatus may be mounted on the suspension apparatus, for example mounted on a side edge of the beam limiter 117, and the like, and the embodiments of the present application are not limited thereto. The image capture apparatus 140 includes one or more cameras, such as a digital camera or an analog camera, or a depth camera, an infrared camera or an ultraviolet camera, or a 3D camera or a 3D scanner, or a red, green and blue (RGB) sensor, an RGB depth (RGB-D) sensor or other devices that can capture color image data of a target object.

In some embodiments, the control apparatus 150 may include a source controller and a detector controller. The source controller is used to command the X-ray source to emit X-rays for image exposure. The detector controller is used to select a suitable detector from among a plurality of detectors and to coordinate the control of various detector functions, such as automatically selecting a corresponding detector according to the position or pose of the subject under examination. Alternatively, the detector controller may perform various signal processing and filtering functions, specifically, for initial adjustment of a dynamic range, interleaving of digital image data, and the like. In some embodiments, the control apparatus may provide power and timing signals for controlling the operation of the X-ray source and the detector.

In some embodiments, the control apparatus may also be configured to use a digitized signal to reconstruct one or more required images and/or determine useful diagnostic information corresponding to a patient, wherein the control apparatus may include one or more dedicated processors, graphics processing units (GPUs), digital signal processors, microcomputers, microcontrollers, application-specific integrated circuits (ASICs), field-programmable gate arrays (FPGAs), or other suitable processing apparatuses.

Certainly, the medical imaging system may also include other numbers, configurations or forms of control apparatuses, for example, the control apparatus may be local (e.g., co-located with one or more X-ray imaging systems 100, e.g., within the same facility and/or the same local network). In other implementations, the control apparatus may be remote, and thus only accessible by means of a remote connection (for example, by means of the Internet or other available remote access technologies). In a specific implementation, the control apparatus may also be configured in a cloud-like means, and may be accessed and/or used in a means that is substantially similar to the means by which other cloud-based systems are accessed and used.

The system 100 also includes a storage apparatus (not shown in the figure). A processor may store the digitized signal in a memory. For example, the memory may include a hard disk drive, a floppy disk drive, a CD-read/write drive, a digital versatile disc (DVD) drive, a flash drive, and/or a solid-state memory. The memory may also be integrated together with the processor to effectively use the footprint and/or meet expected imaging requirements.

The system 100 further includes an input apparatus 160. The input apparatus 160 may include a certain form of operator interface, such as a keyboard, a mouse, a voice-activated control apparatus, a touch screen (which may also be used as a display apparatus described later), a tracking ball or any other suitable input device. An operator may input an operating signal/control signal to the control apparatus by means of the input device.

The system 100 further includes a display apparatus 151 (such as a touch screen or a display screen). The display apparatus 151 may be used to display an operation interface such as a list of subjects under examination, the positioning or exposure configurations of subjects under examination, and images of subjects under examination.

Currently, it is possible to display, on the display apparatus, a captured image obtained by using the image capture apparatus to capture a subject under examination. The captured image is distinguished from the medical image acquired by performing X-ray imaging, and is used for auxiliary positioning or exposure configurations. For example, the auxiliary positioning or exposure configurations include acquiring information of the subject under examination, acquiring a capture protocol, determining information such as a capture dose and posture, performing positioning on the basis of the capture protocol, setting the size and position of an exposure region, etc.

In order to provide convenience for the operator to accurately determine the timing for performing imaging on the subject under examination, embodiments of the present application provide a medical imaging system and a control method for a medical imaging system.

The embodiments of the present application are specifically described below.

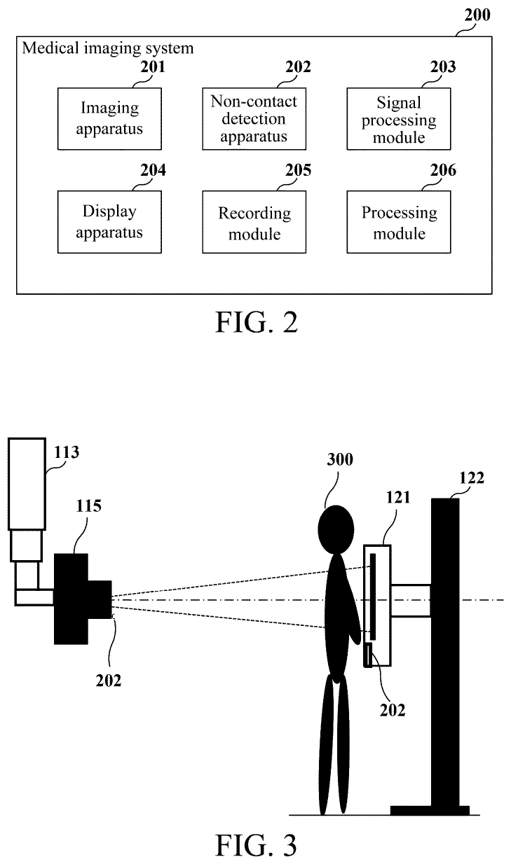

An embodiment of the present application proposes a medical imaging system. FIG. 2 is a schematic diagram of a medical imaging system according to an embodiment of the present application. As shown in FIG. 2, the medical imaging system 200 includes: an imaging apparatus 201, a non-contact detection apparatus 202, a signal processing module 203, and a display apparatus 204.

The imaging apparatus 201 acquires a medical image of a subject under examination. The non-contact detection apparatus 202 may perform non-contact detection on the subject under examination, so as to acquire data related to vital signs of the subject under examination. The signal processing module 203 processes the data to generate information reflecting the vital signs. The display apparatus 204 may display a graphical user interface used to control the imaging apparatus 201, and the information reflecting the vital signs.

In the embodiments of the present application, the information reflecting the vital signs of the subject under examination is displayed. In this way, the operator of the medical imaging system 200 can intuitively obtain the information on the vital signs of the subject under examination, and thus seize an appropriate timing to acquire a medical image of the subject under examination, thereby improving the quality of the medical image. In addition, acquiring the vital signs of the subject under examination by means of the non-contact detection apparatus 202 can improve efficiency and ensure the safety of the subject under examination.

In the descriptions of the embodiments of the present application, the medical imaging system 200 being an X-ray imaging system is used as an example for illustration. However, the present application is not limited thereto. The present application is also applicable to cases where the medical imaging system 200 is another type of medical imaging system; for example, the medical imaging system 200 may be a computerized tomography (CT) system, a positron emission tomography (PET) system, or a magnetic resonance imaging (MRI) system.

In some embodiments, the imaging apparatus 201 may include an X-ray source and a detector. The X-ray source and the detector can cooperate to acquire a medical image of the subject under examination. For descriptions regarding the X-ray source and the detector, reference may be made to the foregoing descriptions. In addition, when the medical imaging system 200 is a computerized tomography (CT) system, a positron emission tomography (PET) system, or a magnetic resonance imaging (MRI) system, the imaging apparatus 201 may have a structure corresponding to the medical imaging system, and reference may be made to related technologies.

For the implementation regarding the display apparatus 204, reference may be made to the description of the display apparatus 151 in the foregoing embodiment corresponding to FIG. 1.

In the present application, the medical imaging system 200 may also have other components. For descriptions regarding the foregoing other components, reference may be made to related descriptions in the foregoing embodiment corresponding to FIG. 1, the repeated portions will not be repeated, and the differences are described in detail below.

In the present application, the data related to the vital signs of the subject under examination may include at least one of the following pieces of data:
Data related to breathing of the subject under examination;
Data related to heartbeats of the subject under examination; and
Data related to the pulse of the subject under examination.

In addition, the present application is not limited thereto, and the data related to the vital signs may also be other data.

In the present application, the non-contact detection apparatus 202 may perform non-contact detection on the subject under examination on the basis of a wireless signal (e.g., an electromagnetic wave, etc.). For example, the non-contact detection apparatus 202 sends a wireless signal to the subject under examination and receives a wireless signal reflected by the subject under examination, so as to perform non-contact detection. Specifically, the non-contact detection apparatus 202 may be a radar. For another example, the non-contact detection apparatus 202 sends a wireless signal to the subject under examination and receives a wireless signal passing through the subject under examination, so as to perform non-contact detection.

In the present application, the signal processing module 203 performs processing on the basis of the data acquired by the non-contact detection apparatus 202 to generate information reflecting the vital signs of the subject under examination. The processing performed on the data by the signal processing module 203 is, for example, at least one of smoothing, filtering, Fourier transform, and the like, so as to obtain information such as a distance, a phase, a position, etc.

In some embodiments, the information on the vital signs may be information on vital signs that change over time.

In some other embodiments, the information on the vital signs may also be information on vital signs that change with frequency. In addition, the signal processing module 203 may further perform processing such as a Fourier transform on the information on the vital signs that change with frequency, so as to convert the information on the vital signs that change with frequency into the information on the vital signs that change over time.

For example, in the case where the non-contact detection apparatus 202 is a radar, the signal processing module 203 performs filtering and fast Fourier transform processing on the data acquired by the non-contact detection apparatus 202 to generate information on a distance that changes over time (such as distance information arranged according to a time sequence). The distance may represent a distance between the non-contact detection apparatus 202 and the surface of the subject under examination.

The distance may reflect a vital sign of the subject under examination. In this way, the information on the distance that changes over time, generated by the signal processing module 203, may be information reflecting the vital sign that changes over time.

For example, when the surface of the subject under examination is the surface of the chest or abdomen, the distance may reflect a breathing state of the subject under examination.

The embodiments of the present application are further described below using the information reflecting the vital signs being information on a breathing state that changes over time as an example.

FIG. 3 is a schematic diagram in which a non-contact detection apparatus performs detection on a subject under examination. As shown in FIG. 3, the non-contact detection apparatus 202 may be disposed on the inner side of the first detector assembly 121 and/or on the tube assembly 115. In addition, when the subject 300 under examination is lying on the examination table 130 (that is, the subject 300 under examination is in a supine position), detection may also be performed merely by means of the non-contact detection apparatus 202 mounted on the tube assembly 115. For descriptions regarding other components in FIG. 3, reference may be made to the descriptions of components with the same reference numerals in FIG. 1.

As shown in FIG. 3, the non-contact detection apparatus 202 may be a radar, which sends a wireless signal to the chest or abdomen of the subject 300 under examination and receives a wireless signal reflected by the body surface of the subject 300 under examination, so as to acquire data related to vital signs of the subject 300 under examination.

The signal processing module 203 processes the data acquired by the non-contact detection apparatus 202 to generate information reflecting the vital signs, for example, to generate information on a distance that changes over time (such as distance information arranged according to a time sequence). The information on the distance that changes over time can reflect information on breathing of the subject 300 under examination that changes over time.

In the present application, the information on the distance that changes over time (namely, the information reflecting the vital signs), generated by the signal processing module 203, may be stored (for example, stored by the recording module 205 described later). In some embodiments, the storage format may be a two-dimensional data format, such as [time, distance]. In some other embodiments, the storage format may be a one-dimensional data format, such as “distance 1, distance 2, distance 3, . . . ”, that is, the distance is recorded every predetermined time period. In this way, the volume of data stored may be reduced. The information may be saved in the form of a digital imaging and communications in medicine (DICOM) header file in a picture archiving and communication system (PACS).

In the present application, the signal processing module 203 may send the generated information on the distance that changes over time (namely, the information reflecting the vital signs) to the display apparatus 204, and the display apparatus 204 can display the information reflecting the vital signs. The display apparatus 204 can display the information reflecting the vital signs in at least one of the following forms: a waveform; a real-time numerical value; an indicator bar, etc.

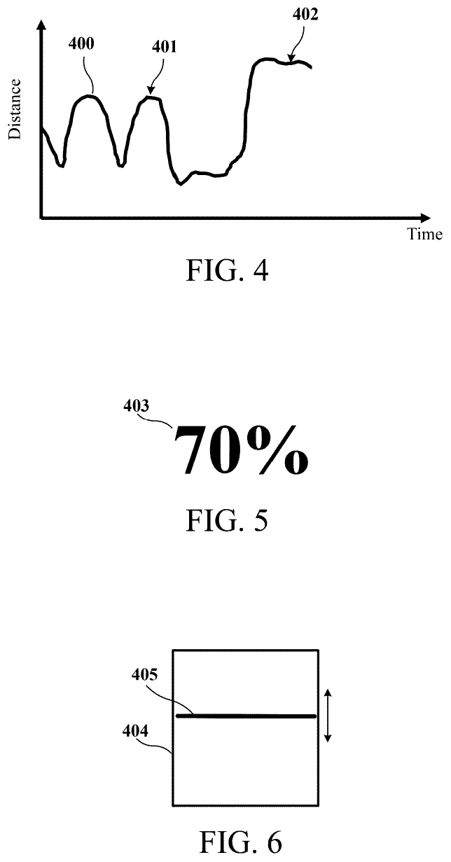

FIG. 4 is a schematic diagram of information reflecting vital signs that is displayed by a display apparatus 204. As shown in FIG. 4, the information reflecting the vital signs may be displayed in the form of a waveform 400, with an abscissa representing time and an ordinate representing distance.

The operator of the medical imaging system 200 may determine an optimal timing to acquire a medical image by observing the waveform 400, thereby improving the quality of the medical image. For example, in the waveform 400, a time point for a wave peak 401 may correspond to a time point when the subject 300 under examination has the maximal inspiratory volume, and a period for a flat waveform portion 402 corresponds to a period in which the subject 300 under examination holds breath after maximum inhalation. Therefore, a medical image with a better imaging effect can be acquired by performing exposure at the time point of the wave peak or in the period of the flat waveform portion. It is easy for the operator to determine the optimal timing to acquire a medical image, especially in examinations without specific inhalation requirements or in the case where the subject 300 under examination is unable to follow breathing instructions.

FIG. 5 is another schematic diagram of information reflecting vital signs that is displayed by a display apparatus 204. As shown in FIG. 5, the information reflecting the vital signs may be displayed in the form of a real-time numerical value. For example, the numerical value 403 in FIG. 5 represents the percentage a current inhalation volume represents of a maximum inhalation volume when the subject 300 under examination is breathing.

FIG. 6 is yet another schematic diagram of information reflecting vital signs that is displayed by a display apparatus 204. As shown in FIG. 6, the information reflecting the vital signs may be displayed in the form of an indicator bar. For example, the indicator bar 404 in FIG. 6 may have an indicator icon 405, and the vertical position of the indicator icon 405 may change with the current inhalation volume of the subject 300 under examination, or the indicator icon 405 may jump up and down with the pulse or heartbeat frequency of the subject 300 under examination.

In the following embodiments of the present application, the information reflecting the vital signs being displayed in the form of a waveform is used as an example for description, and the description is also applicable to other display forms.

In the present application, the display apparatus 204 may also display content such as a graphical user interface used to control the imaging apparatus 201. For example, the graphical user interface may display a control icon that enables the imaging apparatus 201 to acquire a medical image of the subject under examination (for example, enables the imaging apparatus 201 to perform exposure). In addition, the graphical user interface may also display a control icon for adjusting parameters of the imaging apparatus 201, etc.

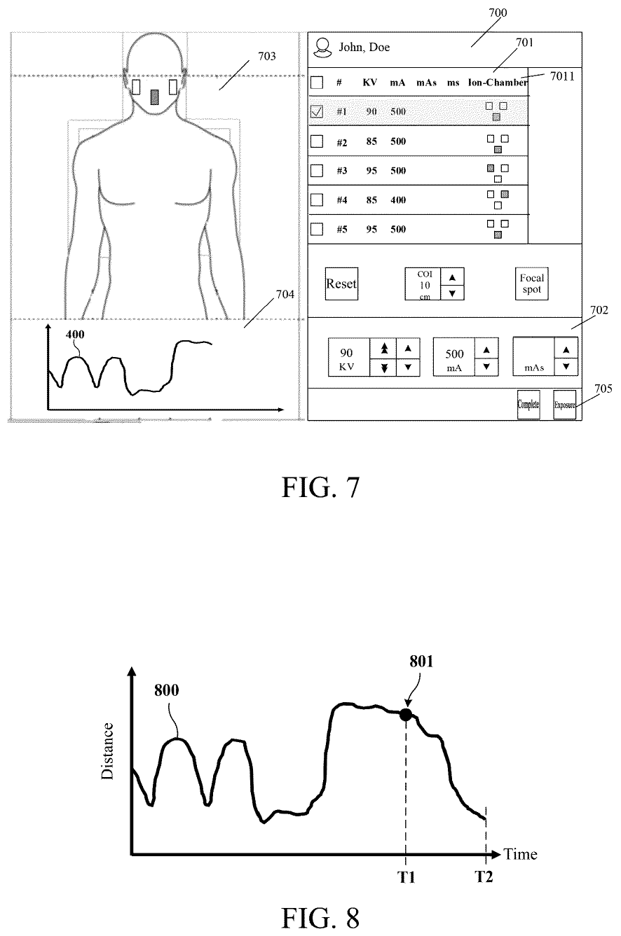

FIG. 7 is a schematic diagram of information reflecting vital signs that is displayed on a graphical user interface. As shown in FIG. 7, the graphical user interface 700 includes a parameter setting display window 701, and the parameter setting display window 701 includes a parameter list 7011. Exposure parameters included in the parameter list 7011 include, for example, a tube voltage (KV), a tube current (mA), milliampere second (mAs), exposure time (ms), an automatic exposure control ionization chamber (Ion-Chamber), etc. The figure shows a parameter list for image stitching, but for different types of exposures, the parameter list is not limited to the arrangement shown in FIG. 7.

In some embodiments, the graphical user interface 700 may further include a parameter adjusting region 702. The parameter adjusting region is used to set and adjust specific numerical values of the parameters.

In some embodiments, the graphical user interface 700 may further include a region 703 for displaying a camera image, the camera image being used to perform positioning, adjust the exposure parameters, etc. before exposure. For example, the camera image may be a real-time video stream captured by the camera apparatus 140 in FIG. 1.

In addition, after exposure, the region 703 may further be used to display a medical image (such as an X-ray image).

In some embodiments, the graphical user interface 700 may further include a region 704 for displaying the information reflecting the vital signs. The information reflecting the vital signs is, for example, in the form of the waveform 400 shown in FIG. 4 or in other forms.

In the present application, the region 704 for displaying the information reflecting the vital signs and the region 703 may be arranged side by side or overlap with each other. In addition, the region 704 may also be a portion of the region 703. In some examples, the region 704 may be displayed in any position of the region 703. For example, the region 704 is displayed in a certain corner of the region 703. Furthermore, the region 704 may also be displayed at any other position on the graphical user interface 700. For example, if the information reflecting the vital signs is displayed in the form of a numerical value, then the region 704 may also be displayed in the parameter adjusting region 702. The position of the region 704 is not limited in the present application.

As shown in FIG. 2, in some embodiments of the present application, the medical imaging system 200 may further include a recording module 205. The recording module 205 may record information on a time point at which the imaging apparatus 201 acquires a medical image (such as a time point for exposure), e.g., may annotate the information on the time point at which the imaging apparatus 201 acquires the medical image in the information reflecting the vital signs. The display apparatus 204 may display the information on the time point at which the imaging apparatus 201 acquires the medical image on a graph.

FIG. 8 is a schematic diagram in which a display apparatus 204 displays information on a time point at which an imaging apparatus 201 acquires a medical image. As shown in FIG. 8, in a waveform 800 where the distance changes over time, a time point corresponding to an icon 801 represents a time point T1 at which the imaging apparatus 201 acquires a medical image (such as a time point at which the imaging apparatus 201 performs exposure).

The information on the time point recorded by the recording module 205 may help to determine whether the medical image is acquired at an appropriate timing (for example, whether the exposure is performed in the case of the maximal inspiratory volume), thereby contributing to reference, as well as analysis and evaluation of the medical image.

In some embodiments, at a predetermined time point after the time point at which the imaging apparatus 201 acquires the medical image, the non-contact detection apparatus 202 may stop performing non-contact detection on the subject 300 under examination. In this way, the volume of data stored can be reduced. For example, in FIG. 8, the predetermined time point after the time point T1 at which the imaging apparatus 201 acquires the medical image is T2, and the non-contact detection apparatus 202 may stop performing non-contact detection on the subject 300 under examination after the time point T2.

The predetermined time point may be a time point at which the imaging apparatus 201 completes image processing of the medical image. In addition, the present application is not limited thereto, and the predetermined time point may also have other meanings.

As shown in FIG. 2, in some embodiments of the present application, the medical imaging system 200 may further include a processing module 206. The processing module 206 may calculate, according to the information reflecting the vital signs, a candidate timing for the imaging apparatus 201 to acquire a medical image, and the display apparatus 204 may display the candidate timing on a graph corresponding to the information reflecting the vital signs. In this way, the operator of the medical imaging system 200 may decide at which time point to acquire a medical image of the subject under examination according to the candidate timing displayed in the display apparatus 204. That is, the patient is unable to hold their breath for pathologic reasons. For example, in the case where the subject 300 under examination is in a natural breathing state but is unable to follow instructions to hold their breath or breathe, the operator can still perform exposure on the basis of the candidate timing displayed in the display apparatus 204 at the time the subject 300 under examination has the maximal inspiratory volume, so as to acquire the medical image.

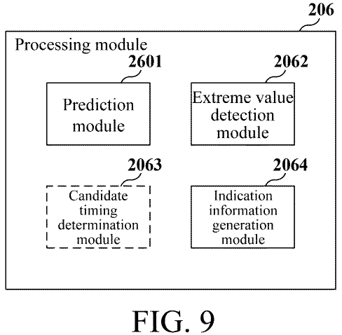

FIG. 9 is a schematic diagram of a processing module 206. As shown in FIG. 9, the processing module 206 includes a prediction module 2601, an extreme value detection module 2602, and a candidate timing determination module 2063.

The prediction module 2061 can predict, on the basis of information reflecting the vital signs within a first time period, information reflecting the vital signs within a second time period after the first time period. The length of the first time period may be preset (for example, set by means of parameters); and the length of the second time period may be preset, or the length of the second time period is determined by the predictive capability of the prediction module 2061.

The prediction module 2061 may predict the information reflecting the vital signs within the second time period on the basis of a long short-term memory (LSTM) neural network.

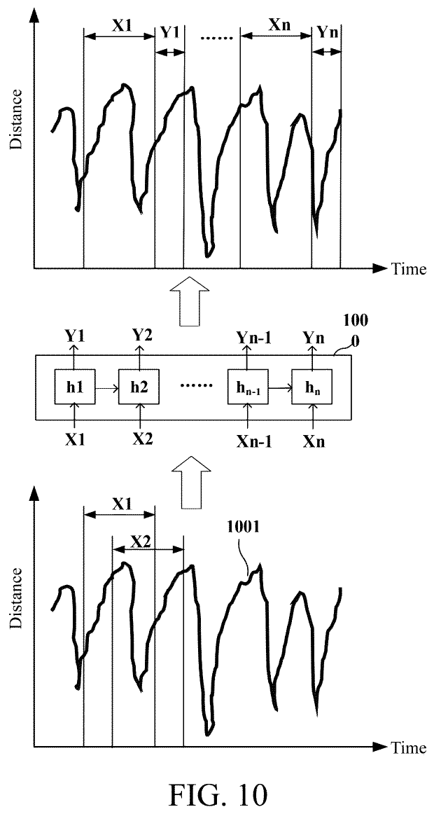

FIG. 10 is a schematic diagram in which information reflecting vital signs within a second time period is predicted on the basis of a long short-term memory (LSTM) neural network.

As shown in FIG. 10, the information reflecting the vital signs that is represented by a waveform 1001 is divided into a plurality of segments (e.g, X1, X2, . . . , Xn−1, Xn), information in each segment is the information reflecting the vital signs within the first time period, the plurality of segments of information are inputted to corresponding hidden layers of a long short term memory neural network 1000, respectively, to form corresponding hidden layer vectors (e.g, h1, h2, . . . , hn−1, hn), and each hidden layer outputs information reflecting the vital signs within a second time period that corresponds to each segment of information (e.g, Y1, Y2, . . . , Yn−1, Yn), wherein Y1 is after X1, . . . , Yn is after Xn, that is, the information within the second time period is predicted on the basis of the information within the first time period. X1, X2, . . . , Xn−1, Xn can be continuously updated with the data outputted by the signal processing module 203, and Y1, Y2, . . . , Yn−1, Yn can also be updated accordingly.

In some examples, the long short-term memory neural network 700 may include a plurality of LSTM layers, and each LSTM layer may include an input unit, a memorizing/forgetting unit, and an output unit. Each LSTM layer further has 10-15 hidden layers, and all of the hidden layers are closely connected to the output unit for outputting the predicted information within the second time period.

In the LSTM neural network: the input unit of each LSTM layer determines which of input information of the layer and output information of hidden layers of a previous LSTM layer will be stored in the memorizing/forgetting unit of the LSTM layer; the memorizing/forgetting unit is responsible for determining which information in the memorizing/forgetting unit of the previous LSTM layer needs to be deleted; and the output unit integrates the input information, generates an output result, and updates corresponding hidden layers. Through information processing performed by the three units in the LSTM layer, the LSTM neural network can continuously update the hidden layers and output the information of the hidden layers as the predicted information.

In addition, in the present application, the actually detected information reflecting the vital signs and the predicted information reflecting the vital signs may further be inputted to the LSTM layer of the LSTM neural network for comparison, so as to correct parameters of the LSTM layer. Consequently the prediction accuracy of the LSTM neural network is improved.

In the present application, the prediction module 2061 can more accurately predict the information reflecting the vital signs within the second time period using the long short-term memory (LSTM) neural network.

In addition, the present application may not be limited to the long short-term memory (LSTM) neural network, and other waveform prediction techniques may be used.

In some embodiments, waveform prediction may be performed in combination with linear and nonlinear equations and predictive parameters using a model-based method, for example:
By using an autoregressive moving average model (ARIMA);
By using a sinusoidal model; or
By using a Kalman Filter.

In some other embodiments, adjustment parameters may be calculated with a neural network using a learning-based method to achieve the purpose of prediction. For example, the types of neural networks that may be used are as follows: an artificial neural network (ANN), a recurrent neural network (RNN), etc.

In the present application, the information within the second time period predicted by the prediction module 2061 (for example, the information within the second time period is displayed in the form of a waveform) may be displayed in a different form from the information within the first time period. For example, different colors and/or different types of lines are used (for example, a solid line and a dashed line are respectively used for display). In addition, with the passage of time, the predicted information (for example, displayed in the form of a waveform) will be overwritten by the actually detected information (for example, displayed in the form of a waveform).

In the present application, the extreme value detection module 2602 detects extreme values (such as maximum values or minimum values) in the information reflecting the vital signs within the second time period and sets the extreme value closest in time to that within the first time period as a candidate extreme value. The extreme value detection module 2602 may use a matched filter to perform peak detection on a predicted waveform. In addition, other methods may also be used for peak detection.

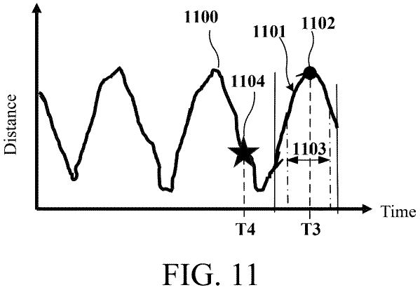

FIG. 11 is a schematic diagram of extreme values. As shown in FIG. 11, in the predicted information 1101 reflecting the vital signs within the second time period, the extreme value (such as the maximum value 1102) closest in time to that within the first time period is detected, and the maximum value 1102 is used as the candidate extreme value. A time point corresponding to the candidate extreme value is at least a portion of the candidate timing.

In the present application, the candidate timing determination module 2603 sets a predetermined time period including the time point corresponding to the candidate extreme value on the basis of the candidate extreme value, and uses the time period as the candidate timing. For example, as shown in FIG. 11, the time period 1103 is used as the candidate timing, and the time period 1103 includes a time point T3 corresponding to the candidate extreme value (such as the maximum value 1102), wherein T3 may be located at the midpoint of the time period 1103, or T3 is before or after the midpoint of the time period 1103.

In addition, the present application is not limited thereto, and the processing module 206 may not have the candidate timing determination module 2603. In this way, the candidate extreme value detected by the extreme value detection module 2602 may be used as the candidate timing.

In the present application, a candidate timing for exposure is indicated by the candidate timing, making it easy for the operator to intuitively observe the candidate timing.

As shown in FIG. 9, in some embodiments, the processing module 206 further includes an indication information generation module 2604. The indication information generation module 2604 may generate indication information at a predetermined time point before the time point corresponding to the candidate extreme value. The display apparatus 204 may display the indication information on a graph (for example, on a waveform).

For example, as shown in FIG. 11, at a time point T4 before the time point T3 corresponding to the maximum value 1102, the indication information 1104 is displayed on the waveform 1100. A time interval between the time point T4 and the time point T3 may be, for example, time required for an exposure action of the imaging apparatus 201. That is, if the operator operates the imaging apparatus 201 at the time point T4, then the actual time point for the imaging apparatus 201 to complete the exposure action is about T3. Therefore, the imaging apparatus 201 can perform exposure at an appropriate timing to acquire a medical image of the subject under examination.

In a specific example, the interval between the time point T4 and the time point T3 is about 1,500 ms.

In addition, the indication information 1104 shown in FIG. 11 is just an example, and the present application is not limited thereto. In some examples, the indication information may also be text or a pop-up window, or the indication information may be embodied by changing the color or shape of the text or the icon. For example, when the indication information is not generated, the icon for controlling exposure on the user interface (such as the icon 705 shown in FIG. 7) may be dimmed or may be in a non-triggerable or non-selectable state, and when the indication information is generated, the indication information may cause the icon for controlling exposure to be lit or cause the icon to be in a triggerable or selectable state.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

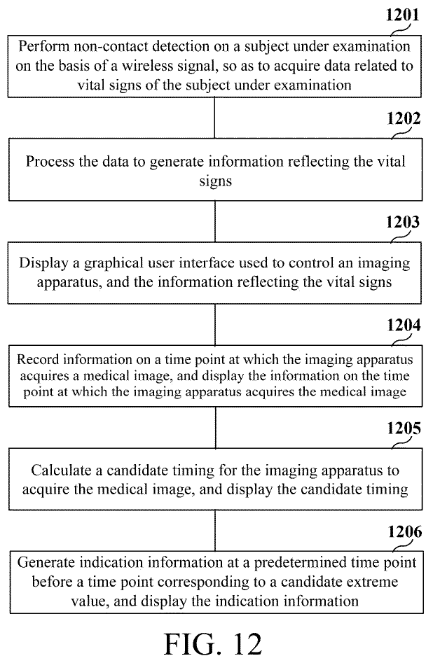

An embodiment of the present application further provides a control method for a medical imaging system. Content which is the same as that of the embodiments of the foregoing aspects will not be repeated. FIG. 12 is a schematic diagram of a control method for a medical imaging system according to an embodiment of the present application. As shown in FIG. 12, the method includes:

Operation 1201, performing non-contact detection on a subject under examination on the basis of a wireless signal, so as to acquire data related to vital signs of the subject under examination;

Operation 1202, processing the data to generate information reflecting the vital signs; and

Operation 1203, displaying a graphical user interface used to control the imaging apparatus and the information reflecting the vital signs.

In some embodiments, the data related to the vital signs of the subject under examination includes at least one of the following pieces of data:
Data related to breathing of the subject under examination;
Data related to heartbeats of the subject under examination; and
Data related to the pulse of the subject under examination.

In some embodiments, as shown in FIG. 12, the method further includes:

Operation 1204, recording information on a time point at which the imaging apparatus acquires the medical image, and displaying the information on the time point at which the imaging apparatus acquires the medical image.

In some embodiments, at a predetermined time point after the time point at which the imaging apparatus acquires the medical image, non-contact detection on the subject under examination is stopped.

In some embodiments, as shown in FIG. 12, the method further includes:

Operation 1205, calculating, according to the information reflecting the vital signs, a candidate timing for the imaging apparatus to acquire the medical image, and displaying the candidate timing.

Calculating a candidate timing for the imaging apparatus to acquire the medical image includes:
Predicting, on the basis of information reflecting the vital signs within a first time period, information reflecting the vital signs within a second time period after the first time period; and
Detecting extreme values in the information reflecting the vital signs within the second time period, and setting the extreme value closest in time to that within the first time period as a candidate extreme value, wherein a time point corresponding to the candidate extreme value is at least a portion of the candidate timing.

In some embodiments, the information reflecting the vital signs within the second time period is predicted on the basis of a long short-term memory (LSTM) neural network.

In some embodiments, as shown in FIG. 12, the method further includes:

Operation 1206, generating indication information at a predetermined time point before the time point corresponding to the candidate extreme value, and displaying the indication information.

It should be noted that FIG. 12 above merely schematically illustrates an embodiment of the present application, but the present application is not limited thereto. For example, the order of execution between operations may be appropriately adjusted. In addition, some other operations may be added or some operations may be omitted. Those skilled in the art could make appropriate variations according to the above content, rather than being limited by the disclosure of FIG. 12 described above.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

The above apparatus and method of the present application can be implemented by hardware, or can be implemented by hardware in combination with software. The present application relates to the foregoing type of computer-readable program. When executed by a logic component, the program causes the logic component to implement the foregoing apparatus or a constituent component, or causes the logic component to implement various methods or steps as described above. The present application further relates to a storage medium for storing the above program, such as a hard disk, a magnetic disk, an optical disk, a DVD, a flash memory, etc.

The method/apparatus described with reference to the embodiments of the present application may be directly embodied as hardware, a software module executed by a processor, or a combination of the two. For example, one or more of the functional block diagrams and/or one or more combinations of the functional block diagrams as shown in the drawings may correspond to either software modules or hardware modules of a computer program flow. The foregoing software modules may respectively correspond to the steps shown in the figures. The foregoing hardware modules may be implemented, for example, by firming the foregoing software modules by using a field-programmable gate array (FPGA).

The software modules may be located in a RAM memory, a flash memory, a ROM memory, an EPROM memory, an EEPROM memory, a register, a hard disk, a removable disk, a CD-ROM, or any storage medium in other forms known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, then the software modules may be stored in the MEGA-SIM card or the large-capacity flash memory apparatus.

One or more of the functional blocks and/or one or more combinations of the functional blocks shown in the drawings may be implemented as a general-purpose processor, a digital signal processor (DSP), an application-specific integrated circuit (ASIC), a field-programmable gate array (FPGA) or other programmable logic devices, a discrete gate or transistor logic device, a discrete hardware assembly, or any appropriate combination thereof, which is used for implementing the functions described in the present application. The one or more functional blocks and/or the one or more combinations of the functional blocks shown in the drawings may also be implemented as a combination of computing equipment, such as a combination of a DSP and a microprocessor, a plurality of microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A medical imaging system, comprising:
an imaging apparatus, which acquires a medical image of a subject under examination;
a non-contact detection apparatus, which performs non-contact detection on the subject under examination on the basis of a wireless signal, so as to acquire data related to vital signs of the subject under examination;
a signal processing module, which processes the data to generate information reflecting the vital signs; and
a display apparatus, which displays a graphical user interface used to control the imaging apparatus, and the information reflecting the vital signs.

2. The medical imaging system according to claim 1, wherein the data related to the vital signs of the subject under examination comprises at least one of the following pieces of data:
data related to breathing of the subject under examination;
data related to heartbeats of the subject under examination; and
data related to the pulse of the subject under examination.

3. The medical imaging system according to claim 1, further comprising:
a recording module, which records information on a time point at which the imaging apparatus acquires the medical image,
wherein the display apparatus displays the information on the time point at which the imaging apparatus acquires the medical image.

4. The medical imaging system according to claim 3, wherein at a predetermined time point after the time point at which the imaging apparatus acquires the medical image, the non-contact detection apparatus stops performing non-contact detection on the subject under examination.

5. The medical imaging system according to claim 1, further comprising:
a processing module, which calculates, according to the information reflecting the vital signs, a candidate timing for the imaging apparatus to acquire the medical image,
wherein the display apparatus displays the candidate timing.

6. The medical imaging system according to claim 5, wherein the processing module comprises:
a prediction module, which predicts, on the basis of information reflecting the vital signs within a first time period, information reflecting the vital signs within a second time period after the first time period; and
an extreme value detection module, which detects extreme values in the information reflecting the vital signs within the second time period, and sets the extreme value closest in time to that within the first time period as a candidate extreme value,
wherein a time point corresponding to the candidate extreme value is at least a portion of the candidate timing.

7. The medical imaging system according to claim 5, further comprising an indication information generation module, which generates indication information at a predetermined time point before a time point corresponding to the candidate extreme value, wherein the display apparatus displays the indication information.

8. The medical imaging system according to claim 5, wherein the prediction module predicts the information reflecting the vital signs within the second time period on the basis of a long short term memory (LSTM) neural network.

9. A control method for a medical imaging system, the medical imaging system having an imaging apparatus used to acquire a medical image of a subject under examination, the method comprising:
performing non-contact detection on the subject under examination on the basis of a wireless signal, so as to acquire data related to vital signs of the subject under examination;
processing the data to generate information reflecting the vital signs; and
displaying a graphical user interface used to control the imaging apparatus, and the information reflecting the vital signs.

10. The control method for the medical imaging system according to claim 9, wherein the data related to the vital signs of the subject under examination comprises at least one of the following pieces of data:
data related to breathing of the subject under examination;
data related to heartbeats of the subject under examination; and
data related to the pulse of the subject under examination.

11. The control method for the medical imaging system according to claim 9, further comprising recording information on a time point at which the imaging apparatus acquires the medical image, and displaying the information on the time point at which the imaging apparatus acquires the medical image.

12. The control method for the medical imaging system according to claim 11, wherein at a predetermined time point after the time point at which the imaging apparatus acquires the medical image, non-contact detection on the subject under examination is stopped.

13. The control method for the medical imaging system according to claim 9, further comprising calculating, according to the information reflecting the vital signs, a candidate timing for the imaging apparatus to acquire the medical image, and displaying the candidate timing.

14. The control method for the medical imaging system according to claim 13, wherein calculating a candidate timing for the imaging apparatus to acquire the medical image comprises:
predicting, on the basis of information reflecting the vital signs within a first time period, information reflecting the vital signs within a second time period after the first time period; and
detecting extreme values in the information reflecting the vital signs within the second time period, and setting the extreme value closest in time to that within the first time period as a candidate extreme value,
wherein a time point corresponding to the candidate extreme value is at least a portion of the candidate timing.

15. The control method for the medical imaging system according to claim 13, further comprising generating indication information at a predetermined time point before a time point corresponding to the candidate extreme value, and displaying the indication information.

16. The control method for the medical imaging system according to claim 13, wherein the information reflecting the vital signs within the second time period is predicted on the basis of a long short term memory (LSTM) neural network.
